SEQUENCE LISTING (44 sequences)

SEQ ID NO: 1 - DNA, 20 bp, Homo sapiens
taatacgagtcactataggg

SEQ ID NO: 2 - DNA, 21 bp, Homo sapiens
taatacgagtcactataggga

SEQ ID NO: 3 - DNA, 32 bp, Homo sapiens
agtagtctcgagcatgtgcagggatcctgaca

SEQ ID NO: 4 - DNA, 35 bp, Homo sapiens
tcgtcgactagttcaccgagcacctccttcaggct

SEQ ID NO: 5 - DNA, 21 bp, Homo sapiens
gccgccataagagccatagaa

SEQ ID NO: 6 - DNA, 23 bp, Homo sapiens
gggtaatttgtctctggctttga

SEQ ID NO: 7 - DNA, 24 bp, Homo sapiens
cgccctttgatatctgctccgcag

SEQ ID NO: 8 - DNA, 89 bp, Homo sapiens
gccgccataagagccatagaggaggttgagggtgttgtgacgccctttgatatctgctccgcagcatcaaagccagagacaaattaccc

SEQ ID NO: 9 - DNA, 22 bp, Homo sapiens
agaggaggttgagggtgttgtg

SEQ ID NO: 10 - DNA, 81 bp, Homo sapiens
agagccatagaggaggttgagggtgttgtgacgccctttgatatctgctccgcagcatcaaagccagagacaaattacccc

SEQ ID NO: 11 - DNA, 23 bp, Homo sapiens
tagaggaggttgagggtgttgtg

SEQ ID NO: 12 - DNA, 81 bp, Homo sapiens
tagagccatagaggaggttgagggtgttgtgacgccctttgatatctgctccgcagcatcaaagccagagacaaattaccc

SEQ ID NO: 13 - DNA, 24 bp, Homo sapiens
atagaggaggttgagggtgttgtg

SEQ ID NO: 14 - DNA, 82 bp, Homo sapiens
atagagccatagaggaggttgagggtgttgtgacgccctttgatatctgctccgcagcatcaaagccagagacaaattaccc

SEQ ID NO: 15 - DNA, 24 bp, Homo sapiens
atagaggaggttgagggtgttgtg

SEQ ID NO: 16 - DNA, 19 bp, Homo sapiens
ccttcaggctcgggcagta

SEQ ID NO: 17 - DNA, 21 bp, Homo sapiens
ccctttgagatctgctccgca

SEQ ID NO: 18 - DNA, 121 bp, Homo sapiens
atagaggaggttgagggtgttgtgacgccctttgagagctgctccgcagcatcaaagccagagacaaattacccctggataggccccaccacgaaccacccctactgcccgagcctgaagg

SEQ ID NO: 19 - DNA, 20 bp, Homo sapiens
aattgggccctctagatgca

SEQ ID NO: 20 - DNA, 22 bp, Homo sapiens
gatatcaaagggcgtcacaaca

SEQ ID NO: 21 - DNA, 23 bp, Homo sapiens
cagggccgccataagagccatag

SEQ ID NO: 22 - DNA, 122 bp, Homo sapiens
aattgggccctctagatgcatgctcgagcatgtgcagggatcctgacacggtactggaggcaggcagggccgccataagagccatagaggaggttgagggtgttgtgacgccctttgagatc

SEQ ID NO: 23 - DNA, 22 bp, Homo sapiens
ttgtgacgccctttgatatctg

SEQ ID NO: 24 - DNA, 23 bp, Homo sapiens
ctggataggccccaccacgaacc

SEQ ID NO: 25 - DNA, 24 bp, Homo sapiens
tccgcagcatcaaagccagagaca

SEQ ID NO: 26 - DNA, 102 bp, Homo sapiens
ttgtgacgccctttgatatctgctccgcagcatcaaagccagagacaaattacccctggataggccccaccacgaaccacccctactgcccgagcctgaagg

SEQ ID NO: 27 - DNA, 34 bp, Homo sapiens
agtagtctcgagtaaatagccctcacccaccaac

SEQ ID NO: 28 - DNA, 29 bp, Homo sapiens
tcgtcgactagtccgagagcgcaggcttc

SEQ ID NO: 29 - DNA, 21 bp, Homo sapiens
aatagccctcacccaccaact

SEQ ID NO: 30 - DNA, 20 bp, Homo sapiens
tccaacggctctgtgtcaga

SEQ ID NO: 31 - DNA, 22 bp, Homo sapiens
ccgttacaggcaagttactgcg

SEQ ID NO: 32 - DNA, 150 bp, Homo sapiens
  misc_feature (119)..(119): n is a, c, g, or t
aatagccctcacccaccaactagccgttacaggcaagttactgcgcgatggcgcaccggacagtccggtgcgccaccggtgcgccaccggtgcgccaccggtgcgccaacggtcacttncaacggctagttctgacacagagccgttgga

SEQ ID NO: 33 - DNA, 22 bp, Homo sapiens
aaatagccctcacccaccaact

SEQ ID NO: 34 - DNA, 151 bp, Homo sapiens
  misc_feature (120)..(120): n is a, c, g, or t
aaatagccctcacccaccaactagccgttacaggcaagttactgcgcgatggcgcaccggacagtccggtgcgccaccggtgcgccaccggtgcgccaccggtgcgccaacggtcacttncaacggctagttctgacacagagccgttgga

SEQ ID NO: 35 - DNA, 21 bp, Homo sapiens
gtccaacggctctgtgtcaga

SEQ ID NO: 36 - DNA, 151 bp, Homo sapiens
  misc_feature (59)..(59): n is a, c, g, or t
aatagccctcacccaccaactagccgttacaggcaagttactgcgcgatgggtcacttnccagtccggtgcgccaccggtgcgccaccggtgcgccaccggtgcgccaacggtcacttncaacggctagttctgacacagagccgttggac

SEQ ID NO: 37 - DNA, 426 bp, Homo sapiens
agtagtctcgagcatgtgcagggatcctgacacggtactgtcatcagagctcgtacacgtccctaggactgtgccatgacgaggcaggcagggccgccataagagccatagaggaggttgctccgtccgtcccggcggtattctcggtatctcctccaacagggtgttgtgacgccctttgatatctgctccgcagcatctcccacaacactgcgggaaactatagacgaggcgtcgtagaaagccagagacaaattacccctggataggccccaccacgtttcggtctctgtttaatggggacctatccggggtggtgcaaccacccctactgcccgagcctgaaggaggtgctcggtgttggtggggatgacgggctcggacttcctccacgagccacaactagtcgacgattgatcagctgct

SEQ ID NO: 38 - DNA, 195 bp, Homo sapiens
tcgagcatgtgcagggatcctgacacggtactggaggcaggcagggccgccataagagccatagaggaggttgagggtgttgtgacgccctttgatatctgctccgcagcatcaaagccagagacaaattacccctggataggccccaccacgaaccacccctactgcccgagcctgaaggaggtgctcggtgaa

SEQ ID NO: 39 - DNA, 247 bp, Homo sapiens
gggcgaattgggccctctagatgcatgctcgagcatgtgcagggatcctgacacggtactggaggcaggcagggccgccataagagccatagaggaggttgagggtgttgtgacgccctttgatatctgctccgcagcatcaaagccagagacaaattacccctggataggccccaccacgaaccacccctactgcccgagcctgaaggaggtgctcggtgaactagtggatccgagctcggtacca

SEQ ID NO: 40 - RNA, 247 nt, Homo sapiens
gggcgaauugggcccucuagaugcaugcucgagcaugugcagggauccugacacgguacuggaggcaggcagggccgccauaagagccauagaggagguugaggguguugugacgcccuuugauaucugcuccgcagcaucaaagccagagacaaauuaccccuggauaggccccaccacgaaccaccccuacugcccgagccugaaggaggugcucggugaacuaguggauccgagcucgguacca

SEQ ID NO: 41 - DNA, 568 bp, Homo sapiens
  misc_feature (253)..(253): n is a, c, g, or t
agtagtctcgagtaaatagccctcacccaccaactagccgtcatcagagctcatttatcgggagtgggtggttgatcggcttacaggcaagttactgcgcgatggcgcaccggacagtccaatgtccgttcaatgacgcgctaccgcgtggcctgtcaggggtgcgccaccggtgcgccaccggtgcgccaccggtgcgcccacgcggtcgccacgcggtggccacgcggtggccacgcgcaacggtcacttncaacggctagttctgacacagagccgtgttgccagtgaangttgccgatcaagactgtgtctcggcatggactcatgacgcaccggacagtgaatagttcactgtccacctgagtactgcgtggcctgtcacttatcaagtgacaggggtgcacaccggacagtccggtgcggtgtccggtgtgccaccacgtgtggcctgtcaggccacgccacaggccacacggtctaaaattcatctccgaagcctgcgctctcggactagtcggattttaagtagaggcttcggacgcgagagcctgatcagcacgatgct

SEQ ID NO: 42 - DNA, 266 bp, Homo sapiens
tcgagtaaatagccctcacccaccaactagccgttacaggcaagttactgcgvgatggcgcaccggacagtccggtgcgccaccggtgcgccaccggtgcgccaccggtgcgccaacggtcacttccaacggctagttctgacacagagccgttggactcatgacgcaccggacagtgaatagttcactgtccggtgcacaccggacagtccggtgcggtgtccggtgtgccactaaaattcatctccgaagcctgcgctctcgga

SEQ ID NO: 43 - DNA, 318 bp, Homo sapiens
  misc_feature (154)..(154): n is a, c, g, or t
gggcgaattgggccctctagatgcatgctcgagtaaatagccctcacccaccaactagccgttacaggcaagttactgcgcgatggcgcaccggacagtccggtgcgccaccggtgcgccaccggtgcgccaccggtgcgccaacggtcacttncaacggctagttctgacacagagccgttggactcatgacgcaccggacagtgaatagttcactgtccggtgcacaccggacagtccggtgcggtgtccggtgtgccactaaaattcatctccgaagcctgcgctctcggactagtgcatccgagctcggtacca

SEQ ID NO: 44 - RNA, 318 nt, Homo sapiens
  misc_feature (154)..(154): n is a, c, g, or u
gggcgaauugggcccucuagaugcaugcucgaguaaauagcccucacccaccaacuagccguuacaggcaaguuacugcgcgauggcgcaccggacaguccggugcgccaccggugcgccaccggugcgccaccggugcgccaacggucacuuncaacggcuaguucugacacagagccguuggacucaugacgcaccggacagugaauaguucacuguccggugcacaccggacaguccggugcgguguccggugugccacuaaaauucaucuccgaagccugcgcucucggacuagugcauccgagcucgguacca

(19) United States
(12) Patent Application Publication
(10) Pub. No.: US 2011/0003309 A1
(43) Pub. Date: Jan. 6, 2011

(54) Non-Competitive Internal Controls for Use in Nucleic Acid Tests

(75) Inventors: Jill Detmer, Kensington, CA (US); Xiaoqiao Jiang, Concord, CA (US); Minh Le, El Cerrito, CA (US); David Sherman, Davis, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS HEALTHCARE DIAGNOSTICS INC., Tarrytown, NY (US)

(21) Appl. No.: 12/922,463

(22) PCT Filed: Mar. 19, 2009
(86) PCT No.: PCT/US09/37593

§ 371 Date: Sep. 14, 2010

Related U.S. Application Data
(62) Division of application No. 61/038,064, filed on Mar. 20, 2008.
Application No. 61/038,064 is a division of application No. 61/054,191, filed on May 19, 2008.

(51) Int. Cl. C12Q 1/68; C07H 21/02; C07H 21/04; C12P 19/34

(52) U.S. Cl. 435/6; 536/23.1; 435/91.5

(57) ABSTRACT

Provided are non-competitive internal controls for use in nucleic acid tests (NATs), which are obtained from the organisms Methanobacterium thermoautrophicum (MET) and Zea mays (Corn). The non-competitive internal controls have utility in DNA and RNA NATs selected from Influenza A, Influenza B, parainfluenza viruses 1 to 4 (PIV-1 to PIV-4), respiratory syncytial virus type A (RSV A), RSV B, human metapneumovirus (hMPV), Chlamydia trachomatis (CT), and Neisseria gonorrhea (GC), Hepatitis B virus (HBV), Hepatitis C virus (HCV), Human Immunodeficiency Virus I (HIV-1), and Severe Acute Respiratory Syndrome (SARS).

### TECHNICAL FIELD

This application relates generally to tools for conducting diagnostic assays and more specifically to internal control sequences for use in nucleic acid tests (NATs) that do not compete with the target nucleic acid sequences.

### BACKGROUND OF THE INVENTION

In order to ensure that nucleic acid tests (NATs) are properly performed, the assays require the presence of internal controls. In diagnostic NATs, the presence of an internal control can guarantee the integrity of the test. Specifically, by including an internal control in a NAT, samples testing positive for the internal control and the target nucleic acid are true positives. By contrast, samples testing only for the internal control are true negatives, samples testing only for the target nucleic acid are true negatives, and samples having no detectable internal control or target are false negatives.

The most commonly used diagnostic assay requiring the presence of internal controls is the polymerase chain reaction (PCR) assay, which is a target amplification assay in its traditional use and a target amplification and quantification assay in its modified use. There are several types of PCR assays: traditional PCR (amplification of DNA); reverse-transcriptase PCR (also known as “RT-PCR”; amplification of DNA using RNA as a starting material), real-time PCR (simultaneous quantification and amplification of DNA); and real-time RT-PCR (simultaneous quantification and amplification of DNA using RNA as a starting material).

In amplification assays, such as PCR assays, typically, one of two types of internal controls is used: competitive internal controls and non-competitive internal controls.

With competitive internal controls, the target and the internal control are amplified with one common set of primers under the same conditions and in the same PCR tube. With competitive internal controls, the internal control nucleic acid is flanked by the same primer sequence that is used to initiate amplification of the target nucleic acid. When the PCR assay is performed correctly, the IC nucleic acid will be detected during post amplification analysis. As is clear from its name, competitive internal controls are based on competition between target DNA and the internal control. For competitive internal controls to be effective, the amount of internal control in the sample tube is critical to the detection limit. A disadvantage of the use of competitive internal controls is based upon its structure, specifically, simultaneous amplification of two different nucleic acid fragments flanked by the same primer sites risks inhibition or enhancement of one or both products depending on the molar ratio, the length, the sequence, and the secondary structure of the nucleic acid fragments. Another disadvantage of competitive internal controls is that they are incapable for use in multiplex assays, which screen multiple targets in a single assay.

With non-competitive internal controls, the target and the internal control are amplified using a different primer set for each; thus, non-competitive internal controls require a PCR in which two reactions with different kinetics proceed simultaneously. Because the kinetics of the two reactions is different, there is no competition for the primers. Non-competitive internal control primer sets currently in use typically target genes other than the target gene (e.g., encoding rRNA), which are present in a sample in higher copy number than the target gene. The most commonly used non-competitive internal control in the art uses primers specific to conserved sequences of 16S and 23S ribosomal DNA. There remains a need in the art for additional non-competitive internal controls that may be prepared for use in multiple assays. An advantage of non-competitive internal controls is that unlike competitive internal controls, non-competitive internal controls they may be stored for use in multiple reactions and also may be used in multiplex reactions. There is a need in the art for such a non-competitive internal control.

### SUMMARY OF THE INVENTION

The present invention overcomes the need in the art for a non-competitive internal control for use in NATs by providing nucleic acid sequences that may be prepared in the lab and stored for use in multiple reactions and in multiplex NATs.

In one aspect of the invention, there is provided a non-competitive internal control for use in nucleic acid tests (NATs) comprising a nucleic acid obtained from an organism selected from Methanobacterium thermoautrophicum (MET) and Zea mays.

In one embodiment of the invention, the non-competitive internal controls are comprised of DNA and are used in DNA NATs selected from the group consisting of Influenza A, Influenza B, parainfluenza viruses 1 to 4 (PIV-1 to PIV-4), respiratory syncytial virus type A (RSV A), RSV B, human metapneumovirus (hMPV), Chlamydia trachomatis (CT), Neisseria gonorrhea (GC [for gonococci]), and Hepatitis B virus (HBV).

In another embodiment of the invention, the non-competitive internal control are comprised of RNA and are used in RNA NATs selected from the group consisting Hepatitis C virus (HCV), Human Immunodeficiency Virus 1 (HIV-1), and Severe Acute Respiratory Syndrome (SARS).

In another aspect of the invention, there is provided, a method of preparing a non-competitive internal control for use in nucleic acid tests (NATs), comprising the steps of: (a) extracting genomic DNA from Methanobacterium thermoautrophicum (MET); (b) generating an amplicon from the genomic DNA of step (a) using forward and reverse primers having at least two restriction enzyme sites and at least one target specific sequence; (c) generating a plasmid by ligating the amplicon of step (b) with a vector sequence having a promoter sequence and restriction enzyme sites that are identical to the restriction enzyme sites of the amplicon; and (d) digesting the plasmid with restriction enzymes corresponding to the restriction enzyme sites of steps (b) and (c) to generate MET internal control DNA.

Where RNA is required, the method further comprises the step of (e): preparing MET internal control RNA from the DNA of step (d).

In a further aspect of the invention, there is provided a method of preparing a non-competitive internal control for use in nucleic acid tests (NATs), comprising the steps of: (a) extracting genomic DNA from Zea Mays (Corn); (b) generating an amplicon from the genomic DNA of step (a) using forward and reverse primers having at least two restriction enzyme sites and target specific sequence; (c) generating a plasmid by ligating the amplicon of step (b) with a vector sequence having a promoter sequence and restriction enzyme sites that are identical to the restriction enzyme sites of the amplicon; (d) digesting the plasmid with restriction enzymes corresponding to the restriction enzyme sites of steps (b) and (c) to generate Corn internal control DNA.

Where RNA is required, the method further comprises the step of: (e) preparing Corn internal control RNA from the DNA of step (d).

In one embodiment of the invention, the at least two restriction enzyme sites of steps (b) and (d) (for both MET and Corn) correspond to the sequences of restriction enzymes XhoI and SpeI.

In another embodiment of the invention, the promoter sequence of step (c) (for both MET and Corn) is a T7 promoter sequence.

In a further embodiment of the invention, the MET or Corn internal control DNA is used as an non-competitive internal control in DNA NATs selected from the group consisting of: Influenza A, Influenza B, parainfluenza viruses 1 to 4 (PIV-1 to PIV-4), respiratory syncytial virus type A (RSV A), RSV B, human metapneumovirus (hMPV), Chlamydia trachomatis (CT), Neisseria gonorrhea (GC), and Hepatitis B virus (HBV).

In yet another embodiment of the invention MET or Corn internal control RNA is used as a non-competitive internal control in RNA NATs selected from the group consisting of: Hepatitis C virus (HCV), Human Immunodeficiency Virus 1 (HIV-1), and Severe Acute Respiratory Syndrome (SARS).

Additional aspects, advantages and features of the invention will be set forth, in part, in the description that follows, and, in part, will become apparent to those skilled in the art upon examination of the following, or may be learned by practice of the invention.

### BRIEF DESCRIPTION OF THE DRAWINGS

FIG. 1 is a schematic diagram of the method for preparing the internal control sequences of the present invention.

FIG. 2 is a graph of the amplification plot for Chlamydia trachomatis (CT) (left curve) and MET IC (right curve) in a single well.

FIG. 3 is a graph of the amplification plot for Neisseria gonorrhea (GC) (left curve) and MET IC (right curve) in a single well.

FIG. 4 is a graph of the amplification plot for Hepatitis C virus (HCV) (left curve) and MET IC (right curve) in a single well.

### DETAILED DESCRIPTION OF THE INVENTION

Set forth below is a description of what are currently believed to be the preferred embodiments and best examples of the claimed invention. Any alternates or modifications in function, purpose, or structure are intended to be covered by the claims of this application.

### DEFINITIONS

In describing and claiming the present invention, the following terminology the following definitions are used for the purpose of describing particular embodiments only, and is not intended to be limiting. As used in this specification and the appended claims, the singular forms “a,” “an,” and “the” include plural referents unless the context clearly dictates otherwise.

The term “non-competitive internal control” refers to an internal control nucleic acid sequence that includes primer sites that are not present in the target nucleic acid sequence.

The term “competitive internal control” refers to an internal control nucleic acid sequence that includes primer sites that are also present in the target nucleic acid sequence.

The terms “FW” and “FP” indicate forward primers and the terms “RV” and “RP” indicate reverse primers. The term “P” when used alone refers to a probe.

The term “PCR primer for the construction of internal control (IC) clone” refers to oligonucleotides that were designed to introduce unique sequences and restriction sites into a newly constructed IC plasmid DNA through overlapping PCR reactions.

The term “amplification primer” (also referred to herein as “primer”) refers to an oligonucleotide that is complementary to DNA or RNA molecules and provides the 3-OH-end of a substrate to which any DNA polymerase can add the nucleotides of a growing DNA chain in the 5 to 3 direction.

The term “detection probe” (also referred to herein as “probe”) refers to an oligonucleotide capable of selectively hybridizing to the amplified target nucleic acid under appropriate conditions. The detection probe may consist of a nucleotide with 5-reporter dye (R) and a 3-quencher dye (Q). A fluorescent reporter dye and fluorophore or a quencher that is either red-shifted fluorescent or non-fluorescent may be covalently linked to the 5-end or 3-end of the oligonucleotide. The detection probe acts as a TAQMAN® (Applied Biosystems, Foster City, Calif.) probe or other detection probes, such as beacons, non-nuclease real time amplification probes during amplification and detection process.

The term “diagnostic target (unknown)” refers to the nucleic acid sequence(s) that the PCR assay has been designed to detect specifically. Examples of these assays include targets, such as for example, Hepatitis B Virus (HBV), Hepatitis C Virus (HCV), and Human Immunodeficiency Virus (HIV).

The term “diagnostic target (known)” refers to the unique DNA or RNA target that is spiked at a known concentration into either the extraction step or amplification mixture used to isolate and amplify the Specific Diagnostic Target whose presence or quantity in the sample is unknown. In addition, unique primers and probes that recognize the unique fragments of RNA or DNA are added into the amplification mixture. These internal controls can be used to monitor the efficiency of the target extraction, amplification, and detection in real time kPCR assays.

As used herein, the term “target amplification” refers to enzyme-mediated procedures that are capable of producing billions of copies of nucleic acid target. Examples of enzyme-mediated target amplification procedures known in the art include PCR, nucleic acid-sequence-based amplification (“NASBA”), transcription-mediated amplification (“TMA”), strand displacement amplification (“SDA”), and ligase chain reaction (“LCR”). The most widely used target amplification procedure is PCR, first described for the amplification of DNA by Mullins et al. in U.S. Pat. No. 4,683,195 and Mullis in U.S. Pat. No. 4,683,202. The PCR procedure is well known to those of ordinary skill in the art. Where the starting material for the PCR reaction is RNA, complementary DNA (“cDNA”) is made from RNA via reverse transcription. A PCR used to amplify RNA products is referred to as reverse transcriptase PCR or “RT-PCR.”

In the PCR technique, a sample of DNA is mixed in a solution with a molar excess of two oligonucleotide primers of 10-30 base pairs each that are prepared to be complementary to the 3′ end of each strand of the DNA duplex; a molar excess of unattached nucleotide bases (i.e., dNTPs); and DNA polymerase, (preferably Taq polymerase, which is stable to heat), which catalyzes the formation of DNA from the oligonucleotide primers and dNTPs. Of the two primers, one is a forward primer that will bind in the 5′-3′ direction to the 3′ end of one strand of the denatured DNA analyte and the other is a reverse primer that will bind in the 3′-5′ direction to the 5′ end of the other strand of the denatured DNA analyte. The solution is heated to 94-96° C. to denature the double-stranded DNA to single-stranded DNA. When the solution cools, the primers bind to the separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. In other words, an extension product synthesized from the forward primer, upon separation, would serve as a template for a complementary extension product synthesized from the reverse primer. Similarly, the extension product synthesized from the reverse primer, upon separation, would serve as a template for a complementary extension product synthesized from the forward primer. In this way, the region of DNA between the primers is selectively replicated with each repetition of the process. Since the sequence being amplified doubles after each cycle, a theoretical amplification of one billion copies may be attained after repeating the process for a few hours; accordingly, extremely small quantities of DNA may be amplified using PCR in a relatively short period of time.

As used herein, the term “amplicon” refers to amplified nucleic acid product, such as for example, amplified PCR product.

Where the starting material for the PCR reaction is RNA, complementary DNA (“cDNA”) is made from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. The enzymes are used in genetic engineering to produce specific cDNA molecules from purified preparations of mRNA. A PCR used to amplify RNA products is referred to as reverse transcriptase PCR or “RT-PCR.”

The terms “real-time PCR” and “real-time RT-PCR,” also known in the art as “kinetic PCR” (“kPCR”) or “kinetic RT-PCR” (“kRT-PCR”), refers to modified PCR assays that are used for simultaneous amplification and quantification of DNA. With real-time PCR, PCR products are detected via a fluorescent signal generated by the coupling of a fluorogenic dye molecule and a quencher moiety to the same or different oligonucleotide substrates. Examples of commonly used probes used in kPCR and kRT-PCR include the following probes: TAQMAN® probes (Applied Biosystems, Foster City, Calif.), Molecular Beacons probes (PHRI, Neward, N.J.), SCORPION® probes (DXS Ltd, Manchester, UK), and SYBR® Green probes (Invitrogen, Carlsbad, Calif.). Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the unhybridized state, the proximity of the fluor and the quench molecules prevents the detection of fluorescent signal from the probe. By contrast, during PCR, when the polymerase replicates a template on which a probe is bound, the 5′-nuclease activity of the polymerase cleaves the probe thus increasing fluorescence with each replication cycle. SYBR® Green probes binds double-stranded DNA and upon excitation emit light; thus as PCR product accumulates, fluorescence increases.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of 80% or more fall within the scope of the present invention.

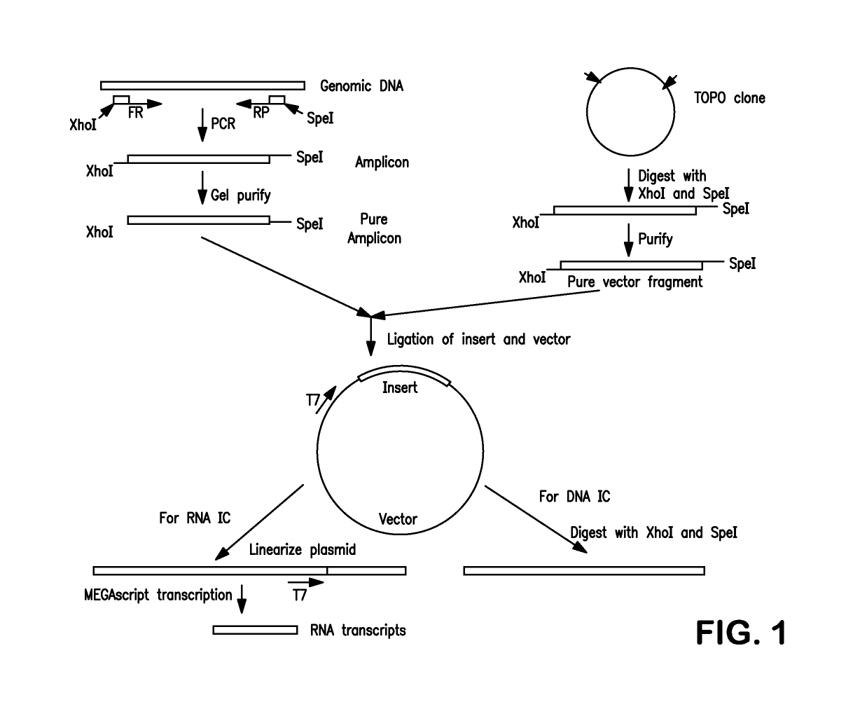

As used herein, the term “cloning” is used to refer to “molecular cloning,” which is a process that creates multiple copies of a nucleic acid sequence (also referred to herein as an “insert”), such as unique genes or selectable genetic markers, from a single copy of the insert. The cloning process typically occurs in a “cloning vector” (also referred to herein as “vector”), which is a DNA molecule, such as a plasmid or viral DNA chromosome, that is capable of replication in a suitable host cell. A “plasmid” is known in the art as a circular double-stranded DNA molecule that is obtained from a bacterial species. A cloning vector typically has one or more suitable sites for the insertion of the nucleic acid sequences. In a successful cloning, the cloning vector is introduced into the host cell and replication of the cloning vector in the host cell results in a transformed host cell, which expresses the nucleic acid sequences that were inserted into the cloning vector. Replication of the cloning vector in the host cell is typically initiated in via a “promoter,” which is a regulatory region of DNA located upstream (towards the 5′ region) of a gene, and which binds RNA polymerase and transcription factors to initiate RNA transcription. Using this procedure and as shown in FIG. 1, the cloning vector can be used as a template to produce an RNA internal control by routine transcription reaction or a DNA internal control by restriction digestion.

As explained in the Background section, when a non-competitive internal control is used in an amplification reaction, such as PCR, different primer sets are used for the internal control and for the target. The use of the non-competitive internal controls thus requires a PCR in which two reactions with different kinetics proceed simultaneously and the kinetics of each reaction are not influenced by competition for the primers.

An advantage of non-competitive internal controls over competitive internal controls is that because non-competitive internal controls are prepared with their own set of primers, they may be used for many different assays in the same laboratory. Another advantage of non-competitive internal controls is that they can be used for multiplex PCR assays. By contrast, competitive internal controls cannot be used with multiplex PCR assays in which several primer pairs are required. As is known to those of skill in the art, multiplex PCR has much usefulness for molecular diagnostics since multiple pathogens producing similar symptoms may be screened simultaneously in a single reaction.

The non-competitive internal controls of the present invention have at least two primer binding sites and at least one probe binding site. As non-competitive controls, the internal controls of the present invention have unique cloning sequences that do not compete with the target nucleic acid sequences. The internal controls are independently designed from the genomes of the organisms Methanobacterium thermoautrophicum (MET) Zea Mays (Corn). Nucleic acids isolated from the organisms are constructed into a plasmid with a cloning vector.

Procedures for cloning nucleic acids are known to those of skill in the art. FIG. 1 shows an exemplary procedure to clone the internal control nucleic acids of the present invention; the procedure set forth in FIG. 1 was used to generate the non-competitive internal controls described in the Examples. As shown in FIG. 1, the isolated genomic DNA is digested with XhoI and SpeI restriction enzymes and the resulting DNA insert is amplified using PCR and purified. Separately, a vector fragment is prepared from the TOPO® Cloning Vector (Invitrogen, Carlsbad, Calif.), which is digested with XhoI and SpeI restriction enzymes to form a vector fragment with XhoI and SpeI sticky ends, which is subsequently purified. The DNA insert and the vector fragment are then ligated by matching the XhoI and SpeI sticky ends to form a plasmid that includes the genomic DNA insert, the vector fragment, and a T7 promoter sequence. The T7 promoter sequence will typically be either a 20-mer T7 promoter sequence 5′-TAA TAC GAG TCA CTA TAG GG-3′ (SEQ ID NO. 1) or a 21-mer T7 promoter sequence 5′-TAA TAC GAG TCA CTA TAG GGA-3′ (SEQ ID NO. 2). The internal control DNA sequences of the present invention are generated by digesting the plasmid with XhoI or SpeI. The internal control RNA sequences are obtained by transcribing the DNA under conditions known to those of skill in the art.

Modifications to the procedures set forth in FIG. 1, such as for example, replacing the T7 promoter with other promoters known in the art, such as the T3 and SP6 promoters, or flanking the MET or Corn inserts with more than one promoter are within the skill level of those in the art. Similarly, it would be within the skill level of one in the art to modify the method set forth herein by replacing the XhoI and SpeI restriction binding sites with binding sites for other suitable restriction enzymes.

The DNA internal controls of the present invention have utility in DNA nucleic acid tests (DATs), including without limitation: Influenza A, Influenza B, parainfluenza viruses 1 to 4 (PIV-1 to PIV-4), respiratory syncytial virus type A (RSV A), RSV B, human metapneumovirus (hMPV), Chlamydia trachomatis (CT), Neisseria gonorrhea (GC), and Hepatitis B virus (HBV).

The RNA internal controls of the present invention have utility in RNA nucleic acid tests (NATs), including without limitation: Hepatitis C virus (HCV), Human Immunodeficiency Virus 1 (HIV-1), and Severe Acute Respiratory Syndrome (SARS).

In practice, it is to be understood that the internal controls of the present invention are included in the same reaction mix as the sample that is being targeted. In one embodiment of the invention, the internal controls are introduced at the step of virus lysis and consequently, can be used to monitor the RNA or DNA target capture or release at the sample preparation step and/or to monitor the target amplification and detection during real time PCR.

Internal Control Fragments and Primer and Probe Sets:

A sequence listing describes the (i) DNA insert during cloning; (ii) resulting complete double-stranded DNA sequence based on purification following restriction enzyme digest; and (iii) sequence of the single-stranded RNA generated from the T7 promoter with attached vector sequences.

Met Nucleic Acid Fragments

Table 1 shows the sequences of the forward (FP) and reverse (RP) primers that are used to extract nucleic acid fragments from the M. thermoautrophicum (MET) genome, which are used to clone the MET internal controls (MET IC) of the present invention. The primers are designed with restriction enzymes binding sites (highlighted in bold) and target specific binding sites (underlined). As shown therein, the forward primer is designed with an XhoI restriction enzyme sequence (C/TCGAG) and the reverse primer is designed with a SpeI restriction enzyme sequence (A/CTAGT).

TABLE 1

| Frag- | | |
|---|---|---|
| ment | | |
| Prim- | | |
| ers | Sequence 5-3 | Strands |
| MET IC | AGTAGTCTCGAGCATGTGCAGGGATCCTGACA | (+) |
| FP | (SEQ ID. NO. 3) | |
| MET IC | TCGTCGACTAGTTCACCGAGCACCTCCTTCAGGCT | (−) |
| RP | (SEQ ID NO. 4) | |

As indicated above, the internal controls of the present invention include at least two primer binding sites and at least one probe binding site. Tables 2 to 8 set forth various forward and reverse amplification primer sequences and detection probe binding sequences that can be used to generate MET IC Amplicons, which are also provided in the tables. The amplicons set forth in Tables 2 to 8 can be used to prepare non-competitive controls for use in nucleic acid diagnostic tests for the following disease states: Influenza A, Influenza B, parainfluenza viruses 1 to 4 (PIV-1 to PIV-4), respiratory syncytial virus type A (RSV A), RSV B, human metapneumovirus (hMPV), Chlamydia trachomatis (CT), Neisseria gonorrhea (GC), Hepatitis B virus (HBV), Hepatitis C virus (HCV), Human Immunodeficiency Virus 1 (HIV-1), and Severe Acute Respiratory Syndrome (SARS).

TABLE 2

| Set 1 | Sequence 5-3 | Strands |
|---|---|---|
| MET-FW01 | GCCGCCATAAGAGCCATAGAA | (+) |
| | (SEQ ID NO. 5) | |
| MET-RV01 | GGGTAATTTGTCTCTGGCTTTGA | (−) |
| | (SEQ ID NO. 6) | |
| MET-P01 | CGCCCTTTGATATCTGCTCCGCAG | (+) |
| | (SEQ ID NO. 7) | |

| | | | | |
|---|---|---|---|---|
| MET IC | 1 | GCCGCCATAA | GAGCCATAGA | GGAGGTTGAG |
| Amplicon | 31 | GGTGTTGTGA | CGCCCTTTGA | TATCTGCTCC |
| (96 pbs) | 61 | GCAGCATCAA | AGCCAGAGAC | AAATTACCC |

(SEQ ID No. 8)

TABLE 3

| Set 2 | Sequence 5-3 | Strands |
|---|---|---|
| MET-FW02 | AGAGGAGGTTGAGGGTGTTGTG | (+) |
| | (SEQ ID NO. 9) | |
| MET-RV01 | GGGTAATTTGTCTCTGGCTTTGA | (−) |
| | (SEQ ID NO. 6) | |
| MET-P02 | CGCCCTTTGATATCTGCTCCGCAG | (+) |
| | (SEQ ID NO. 7) | |

| | | | | |
|---|---|---|---|---|
| Set 2 | 1 | AGAGCCATAG | AGGAGGTTGA | GGGTGTTGTG |
| Amplicon | 31 | ACGCCCTTTG | ATATCTGCTC | CGCAGCATCA |
| (80 bps) | 61 | AAGCCAGAGA | CAAATTACCC | C |

(SEQ ID NO. 10)

TABLE 4

| Set 3 | Sequence 5-3 | Strands |
|---|---|---|
| MET-FW03 | TAGAGGAGGTTGAGGGTGTTGTG | (+) |
| | (SEQ ID NO. 11) | |
| MET-RV01 | GGGTAATTTGTCTCTGGCTTTGA | (−) |
| | (SEQ ID NO. 6) | |
| MET-P02 | CGCCCTTTCATATCTGCTCCGCAG | (+) |
| | (SEQ ID NO. 7) | |

| | | | | |
|---|---|---|---|---|
| Set 3 | 1 | TAGAGCCATA | GAGGAGGTTG | AGGGTGTTGT |
| Amplicon | 31 | GACGCCCTTT | GATATCTGCT | CCGCAGCATC |
| (81 bps) | 61 | AAAGCCAGAG | ACAAATTACC | C |

(SEQ ID NO. 12)

TABLE 5

| Set 4 | Sequence 5-3 | Strands |
|---|---|---|
| MET-FW04 | ATAGAGGAGGTTGAGGGTGTTGTG | (+) |
| | (SEQ ID NO. 13) | |
| MET-RV01 | GGGTAATTTGTCTCTGGCTTTGA | (−) |
| | (SEQ ID NO. 6) | |
| MET P02 | CGCCCTTTCATATCTGCTCCGCAG | (+) |
| | (SEQ ID NO. 7) | |

| | | |
|---|---|---|
| Set 4 Amplicon | 1 | ATAGAGCCAT AGAGGAGGTT GAGGGTGTTG |
| (82 bps) | 31 | TGACGCCCTT TGATATCTGC TCCGCAGCAT |
| | 61 | CAAAGCCAGA GACAAATTAC CC |

(SEQ ID NO. 14)

TABLE 6

| Set 5 | Sequence 5-3 | Strands |
|---|---|---|
| MET-FW05 | ATAGAGGAGGTTGAGGGTGTTGTG | (+) |
| | (SEQ ID NO. 15) | |
| MET-RV05 | CCTTCAGGCTCGGGCAGTA | (−) |
| | (SEQ ID NO. 16) | |
| MET-P05 | CCCTTTGAGATCTGCTCCGCA | (+) |
| | (SEQ ID NO. 17) | |

| | | |
|---|---|---|
| Set 5 Amplicon | 1 | ATAGAGGAGG TTGAGGGTGT TGTGACGCCC |
| (121 bps) | 31 | TTTGAGAGCT GCTCCGCAGC ATCAAAGCCA |
| | 61 | GAGACAAATT ACCCCTGGAT AGGCCCCACC |
| | 91 | ACGAACCACC CCTACTGCCC GAGCCTGAAG |
| | 121 | G |

(SEQ ID NO. 18)

TABLE 7

| Set 6 | Sequence 5-3 | Strands |
|---|---|---|
| MET-FW06 | AATTGGGCCCTCTAGATGCA | (+) |
| | (SEQ ID NO. 19) | |
| MET-RV06 | GATATCAAAGGGCGTCACAACA | (−) |
| | (SEQ ID NO. 20) | |
| MET-P06 | CAGGGCCGCCATAAGAGCCATAG | (+) |
| | (SEQ ID NO. 21) | |

| | | |
|---|---|---|
| Set 6 | 1 | AATTGGGCCC TCTAGATGCA TGCTCGAGCA |
| Amplicon | 31 | TGTGCAGGGA TCCTGACACG GTACTGGAGG |
| (122 bps) | 61 | CAGGCAGGGC CGCCATAAGA GCCATAGAGG |
| (Vector | 91 | AGGTTGAGGG TGTTGTGACG CCCTTTGAGA |
| sequence is | 121 | TC |

| | |
|---|---|
| highlighted | (SEQ ID NO. 22) |
| in bold | |
| underlining) | |

TABLE 8

| Set 7 | Sequence 5-3 | Strands |
|---|---|---|
| MET-FW07 | TTGTGACGCCCTTTGATATCTG | (+) |
| | (SEQ ID NO. 23) | |
| MET-RV05 | CCTTCAGGCTCGGGCAGTA | (−) |
| | (SEQ ID NO. 16) | |
| METP07a | CTGGATAGGCCCCACCACGAACC | (+) |
| | (SEQ ID NO. 24) | |
| METP07b | TCCGCAGCATCAAAGCCAGAGACA | |
| | (SEQ ID NO. 25) | |

| | | |
|---|---|---|
| Set 7 Amplicon | 1 | TTGTGACGCC CTTTGATATC TGCTCCGCAG |
| (102 bps) | 31 | CATCAAAGCC AGAGACAAAT TACCCCTGGA |
| | 61 | TAGGCCCCAC CACGAACCAC CCCTACTGCC |
| | 91 | CGAGCCTGAA GG |

(SEQ ID NO. 26)

Corn Nucleic Acid Fragments

Table 9 shows the sequences of the forward (FP) and reverse (RP) primers that are used to extract nucleic acid fragments from the Z. mays (Corn) genome, which are used to clone the Corn internal controls (Corn IC) of the present invention. The primers are designed with restriction enzymes binding sites (highlighted in bold) and target specific binding sites (underlined). As shown therein, the forward primer is designed with an XhoI restriction enzyme sequence (C/TCGAG) and the reverse primer is designed with a SpeI restriction enzyme sequence (A/CTAGT).

TABLE 9

| Fragment | | |
|---|---|---|
| Primers | Sequence 5-3 | Strands |
| Corn 1 | AGTAGTCTCGAGTAAATAGCCCTCACCCACCAAC | (+) |
| IC FP | (SEQ ID. NO. 27) | |
| Corn 1 | TCGTCGACTAGTCCGAGAGCGCAGGCTTC | (−) |
| IC RP | (SEQ ID NO. 28) | |

As indicated above, the internal controls of the present invention include at least two primer binding sites and at least one probe binding site. Tables 10 to 12 set forth various forward and reverse amplification primer sequences and detection probe binding sequences that can be used to generate Corn IC Amplicons which are also provided in the tables. The amplicons set forth in Tables 10 to 12 can be used to prepare non-competitive controls for use in nucleic acid diagnostic tests for the following disease states: Influenza A, Influenza B, parainfluenza viruses 1 to 4 (PIV-1 to PIV-4), respiratory syncytial virus type A (RSV A), RSV B, human metapneumovirus (hMPV), Chlamydia trachomatis (CT), Neisseria gonorrhea (GC), Hepatitis B virus (HBV), Hepatitis C virus (HCV), Human Immunodeficiency Virus 1 (HIV-1), and Severe Acute Respiratory Syndrome (SARS).

TABLE 10

| Set 1 | Sequence 5-3 | Strands |
|---|---|---|
| Corn1 IC FP1 | AATAGCCCTCACCCACCAACT | (+) |
| | (SEQ ID NO. 29) | |
| Corn1 IC RP1 | TCCAACGGCTCTGTGTCAGA | (−) |
| | (SEQ ID NO. 30) | |
| Corn1 IC | CCGTTACAGGCAAGTTACTGCG | (+) |
| Probe 1 | (SEQ ID NO. 31) | |

| | | |
|---|---|---|
| Corn IC | 1 | AATAGCCCTC ACCCACCAAC TAGCCGTTAC |
| Amplicon | 31 | AGGCAAGTTA CTGCGCGATG GCGCACCGGA |
| (150 pbs) | 61 | CAGTCCGGTG CGCCACCGGT GCGCCACCGG |
| | 91 | TGCGCCACCG GTGCGCCAAC GGTCACTTNC |
| | 121 | AACGGCTAGT TCTGACACAG AGCCGTTGGA |

(SEQ ID NO. 32)

TABLE 11

| Set 2 | Sequence 5-3 | Strands |
|---|---|---|
| Corn1 IC FP2 | AAATAGCCCTCACCCACCAACT | (+) |
| | (SEQ ID NO. 33) | |
| Corn1 IC RP1 | TCCAACGGCTCTGTGTCAGA | (−) |
| | (SEQ ID NO. 30) | |
| Corn1 IC | CCGTTACAGGCAAGTTACTGCG | (+) |
| Probe 1 | (SEQ ID NO. 31) | |

| | | |
|---|---|---|
| Set 2 Amplicon | 1 | AAATAGCCCT CACCCACCAA CTAGCCGTTA |
| (151 pbs) | 31 | CAGGCAAGTT ACTGCGCGAT GGCGCACCGG |
| | 61 | ACAGTCCGGT GCGCCACCGG TGCGCCACCG |
| | 91 | GTGCGCCACC GGTGCGCCAA CGGTCACTTN |
| | 121 | CAACGGCTAG TTCTGACACA GAGCCGTTGG |
| | 151 | A |

(SEQ ID NO. 34)

TABLE 12

| Set 3 | Sequence 5-3 | Strands |
|---|---|---|
| Corn1 IC FP1 | AATAGCCCTCACCCACCAACT | (+) |
| | (SEQ ID NO. 29) | |
| Corn1 IC RP3 | GTCCAACGGCTCTGTGTCAGA | (−) |
| | (SEQ ID NO. 35) | |
| Corn1 IC | CCGTTACAGGCAAGTTACTGCG | (+) |
| Probe 1 | (SEQ ID NO. 31) | |

| | | |
|---|---|---|
| Set 3 Amplicon | 1 | AATAGCCCTC ACCCACCAAC TAGCCGTTAC |
| (151 pbs) | 31 | AGGCAAGTTA CTGCGCGATG GGTCACTTNC |
| | 61 | CAGTCCGGTG CGCCACCGGT GCGCCACCGG |
| | 91 | TGCGCCACCG GTGCGCCAAC GGTCACTTNC |
| | 121 | AACGGCTAGT TCTGACACAG AGCCGTTGGA |
| | 151 | C |

(SEQ ID NO. 36)

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents and publications mentioned herein are incorporated by reference in their entireties.

### EXPERIMENTAL

The following examples are put forth so as to provide those of ordinary skill in the art with a complete disclosure and description of how to make and use the compositions of the invention. The examples are intended as non-limiting examples of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

General Protocols:

The following protocols, apparatus, and kits were used to carry out the following Examples.

Extraction of Genomic DNA: native target purified by manual Qiagen (Valencia, Calif.) sample preparation kit.

PCR Conditions and Apparatus: PCR was conducted on an MJ Research (Ramsey, Minn.) instrument using the following thermoprofile:

| | | |
|---|---|---|
| 95° 10 min - 1 step | | |
| 95° 15 sec | | |
| 60° 15 sec | {close oversize brace} | repeated for 30 cycles |
| 68° 1 min | | |
| 72° 10 min | | |
| 4° overnight (until removed) | | |

Purification of PCR Product: PCR product was purified using a Qiagen kit (Valencia, Calif.)

Cloning Procedure: Cloning was carried out using the procedure set forth in the product insert for the Invitrogen cloning protocol using a TOPO® Cloning vector (Carlsbad, Calif.).

Ligation Procedure: Ligation of DNA fragments to the TOPO® Cloning vector was carried out using the instructions for Invitrogen T4 ligase (Carlsbad, Calif.).

Purification of Vector Fragment: Purification of vector was carried out using Clontech NUCLEOSPIN® RNA Purification Kit (Mountain View, Calif.).

RNA Transcription Protocol: Transcription was carried out according to the instructions in the product insert for the Ambion T7 MEGASCRIPT® kit (Austin, Tex.). Purification of the RNA was carried out according to the instructions in the Qiagen RNEASY® mini kit (Valencia, Calif.).

### Example 1

### Preparation of MET IC DNA Insert Sequence

Genomic DNA was extracted from a MET sample. The DNA insert was prepared by running a PCR on the genomic DNA with the fragment primers of Table 1. The following sequence is the sequence for the MET IC PCR Product (213 bp) (SEQ ID NO. 37). The XhoI and SpeI restriction enzyme sites are identified with bold underlining.

| | |
|---|---|
| 1 | AGTAGTCTCG AGCATGTGCA GGGATCCTGA CACGGTACTG |
| | TCATCAGAGC TCGTACACGT CCCTAGGACT GTGCCATGAC |
| 41 | GAGGCAGGCA GGGCCGCCAT AAGAGCCATA GAGGAGGTTG |
| | CTCCGTCCGT CCCGGCGGTA TTCTCGGTAT CTCCTCCAAC |
| 81 | AGGGTGTTGT GACGCCCTTT GATATCTGCT CCGCAGCATC |
| | TCCCACAACA CTGCGGGAAA CTATAGACGA GGCGTCGTAG |
| 121 | AAAGCCAGAG ACAAATTACC CCTGGATAGG CCCCACCACG |
| | TTTCGGTCTC TGTTTAATGG GGACCTATCC GGGGTGGTGC |
| 161 | AACCACCCCT ACTGCCCGAG CCTGAAGGAG GTGCTCGGTG |
| | TTGGTGGGGA TGACGGGCTC GGACTTCCTC CACGAGCCAC |
| 201 | AACTAGTCGA CGA |
| | TTGATCAGCT GCT |

### Example 2

### Purified MET IC DNA Insert Sequence

The following sequence is the purified 195 by dsDNA sequence following restriction enzyme digestion at the sites identified above (SEQ ID NO. 38):

| | |
|---|---|
| 1 | TCGAGCATGT GCAGGGATCC TGACACGGTA CTGGAGGCAG |
| 41 | GCAGGGCCGC CATAAGAGCC ATAGAGGAGG TTGAGGGTGT |
| 81 | TGTGACGCCC TTTGATATCT GCTCCGCAGC ATCAAAGCCA |
| 121 | GAGACAAATT ACCCCTGGAT AGGCCCCACC ACGAACCACC |
| 161 | CCTACTGCCC GAGCCTGAAG GAGGTGCTCG GTGAA |

### Example 3

### MET IC DNA Transcript Sequence

A plasmid was prepared by ligating the purified MET IC DNA insert sequence of Example 2 to a purified vector fragment and adding a T7 promoter sequence. The purified vector fragment was isolated from a TOPO® Cloning vector (Invitrogen, Carlsbad, Calif.) via digestion with the restriction enzymes XhoI and SpeI. The plasmid was formed by matching the XhoI and SpeI sticky ends of the DNA insert and the vector. FIG. 1 shows a schematic of the cloning process.

The resultant plasmid was linearized with Xho1 and SpeI to generate the following 247 by MET IC DNA transcript sequence. The vector sequences are highlighted with bold underlining (SEQ ID NO. 39):

| | |
|---|---|
| 1 | GGGCGAATTG GGCCCTCTAG ATGCATGCTC GAGCATGTGC |
| 41 | AGGGATCCTG ACACGGTACT GGAGGCAGGC AGGGCCGCCA |
| 81 | TAAGAGCCAT AGAGGAGGTT GAGGGTGTTG TGACGCCCTT |
| 121 | TGATATCTGC TCCGCAGCAT CAAAGCCAGA GACAAATTAC |
| 161 | CCCTGGATAG GCCCCACCAC GAACCACCCC TACTGCCCGA |
| 201 | GCCTGAAGGA GGTGCTCGGT GAACTAGTGG ATCCGAGCTC |
| 241 | GGTACCA |

### Example 4

### MET IC RNA Transcript Sequence

The following sequence is the 247 by MET IC RNA transcript sequence prepared from the DNA sequence of Example 3. The vector sequences are highlighted with bold underlining (SEQ ID NO. 40):

| | |
|---|---|
| 1 | GGGCGAAUUG GGCCCUCUAG AUGCAUGCUC GAGCAUGUGC |
| 41 | AGGGAUCCUG ACACGGUACU GGAGGCAGGC AGGGCCGCCA |
| 81 | UAAGAGCCAU AGAGGAGGUU GAGGGUGUUG UGACGCCCUU |
| 121 | UGAUAUCUGC UCCGCAGCAU CAAAGCCAGA GACAAAUUAC |
| 161 | CCCUGGAUAG GCCCCACCAC GAACCACCCC UACUGCCCGA |
| 201 | GCCUGAAGGA GGUGCUCGGU GAACUAGUGG AUCCGAGCUC |
| 241 | GGUACCA |

### Example 5

### Preparation of Corn IC Insert Sequence

Genomic DNA was extracted from a Z. mays (Corn) sample. The DNA insert was prepared by running a PCR on the genomic DNA with the fragment primers of Table 9. The following sequence is the sequence for the Corn IC PCR Product (284 bp) (SEQ ID NO. 41). The XhoI and SpeI restriction enzyme sites are identified with bold underlining.

| | |
|---|---|
| 1 | AGTAGTCTCG AGTAAATAGC CCTCACCCAC CAACTAGCCG |
| | TCATCAGAGC TCATTTATCG GGAGTGGGTG GTTGATCGGC |
| 41 | TTACAGGCAA GTTACTGCGC GATGGCGCAC CGGACAGTCC |
| | AATGTCCGTT CAATGACGCG CTACCGCGTG GCCTGTCAGG |
| 81 | GGTGCGCCAC CGGTGCGCCA CCGGTGCGCC ACCGGTGCGC |
| | CCACGCGGTC GCCACGCGGT GGCCACGCGG TGGCCACGCG |
| 121 | CAACGGTCAC TTNCAACGGC TAGTTCTGAC ACAGAGCCGT |
| | GTTGCCAGTG AANGTTGCCG ATCAAGACTG TGTCTCGGCA |
| 161 | TGGACTCATG ACGCACCGGA CAGTGAATAG TTCACTGTCC |
| | ACCTGAGTAC TGCGTGGCCT GTCACTTATC AAGTGACAGG |
| 201 | GGTGCACACC GGACAGTCCG GTGCGGTGTC CGGTGTGCCA |
| | CCACGTGTGG CCTGTCAGGC CACGCCACAG GCCACACGGT |
| 241 | CTAAAATTCA TCTCCGAAGC CTGCGCTCTC GGACTAGTCG |
| | GATTTTAAGT AGAGGCTTCG GACGCGAGAG CCTGATCAGC |
| 281 | ACGA |
| | TGCT |

### Example 6

### Purified Corn IC DNA Insert Sequence

The following sequence is the purified 266 by dsDNA sequence following restriction enzyme digestion at the sites identified above (SEQ ID NO. 42):

| | |
|---|---|
| 1 | TCGAGTAAAT AGCCCTCACC CACCAACTAG CCGTTACAGG |
| 41 | CAAGTTACTG CGVGATGGCG CACCGGACAG TCCGGTGCGC |
| 81 | CACCGGTGCG CCACCGGTGC GCCACCGGTG CGCCAACGGT |
| 121 | CACTTCCAAC GGCTAGTTCT GACACAGAGC CGTTGGACTC |
| 161 | ATGACGCACC GGACAGTGAA TAGTTCACTG TCCGGTGCAC |
| 201 | ACCGGACAGT CCGGTGCGGT GTCCGGTGTG CCACTAAAAT |
| 241 | TCATCTCCGA AGCCTGCGCT CTCGGA |

### Example 7

### Corn IC DNA Transcript Sequence

A plasmid was prepared by ligating the purified MET Corn IC DNA insert sequence of Example 6 to a purified vector fragment with sticky end restriction sites and a T7 promoter sequence. The purified vector fragment was isolated from a TOPO® Cloning vector (Invitrogen, Carlsbad, Calif.) via digestion with the restriction enzymes XhoI and SpeI. The plasmid was formed by matching the XhoI and SpeI sticky ends of the DNA insert and the vector. FIG. 1 shows a schematic of the cloning process.

The resultant plasmid was linearized with Xho1 and SpeI to generate the following 318 by Corn IC DNA transcript sequence. The vector sequences are highlighted with bold underlining (SEQ ID NO. 43):

| | |
|---|---|
| 1 | GGGCGAATTG GGCCCTCTAG ATGCATGCTC GAGTAAATAG |
| 41 | CCCTCACCCA CCAACTAGCC GTTACAGGCA AGTTACTGCG |
| 81 | CGATGGCGCA CCGGACAGTC CGGTGCGCCA CCGGTGCGCC |
| 121 | ACCGGTGCGC CACCGGTGCG CCAACGGTCA CTTNCAACGG |
| 161 | CTAGTTCTGA CACAGAGCCG TTGGACTCAT GACGCACCGG |
| 201 | ACAGTGAATA GTTCACTGTC CGGTGCACAC CGGACAGTCC |
| 241 | GGTGCGGTGT CCGGTGTGCC ACTAAAATTC ATCTCCGAAG |
| 281 | CCTGCGCTCT CGGACTAGTG CATCCGAGCT CGGTACCA |

### Example 8

### Corn IC RNA Transcript Sequence

The following sequence is the 318 by Corn IC RNA transcript sequence prepared from the DNA sequence of Example 7. The vector sequences are highlighted with bold underlining (SEQ ID NO. 44):

| | |
|---|---|
| 1 | GGGCGAAUUG GGCCCUCUAG AUGCAUGCUC GAGUAAAUAG |
| 41 | CCCUCACCCA CCAACUAGCC GUUACAGGCA AGUUACUGCG |
| 81 | CGAUGGCGCA CCGGACAGUC CGGUGCGCCA CCGGUGCGCC |
| 121 | ACCGGUGCGC CACCGGUGCG CCAACGGUCA CUUNCAACGG |
| 161 | CUAGUUCUGA CACAGAGCCG UUGGACUCAU GACGCACCGG |
| 201 | ACAGUGAAUA GUUCACUGUC CGGUGCACAC CGGACAGUCC |
| 241 | GGUGCGGUGU CCGGUGUGCC ACUAAAAUUC AUCUCCGAAG |
| 281 | CCUGCGCUCU CGGACUAGUG CAUCCGAGCU CGGUACCA |

### Experiment 9

### Use of MET IC in CT, GC, and HCV Assays

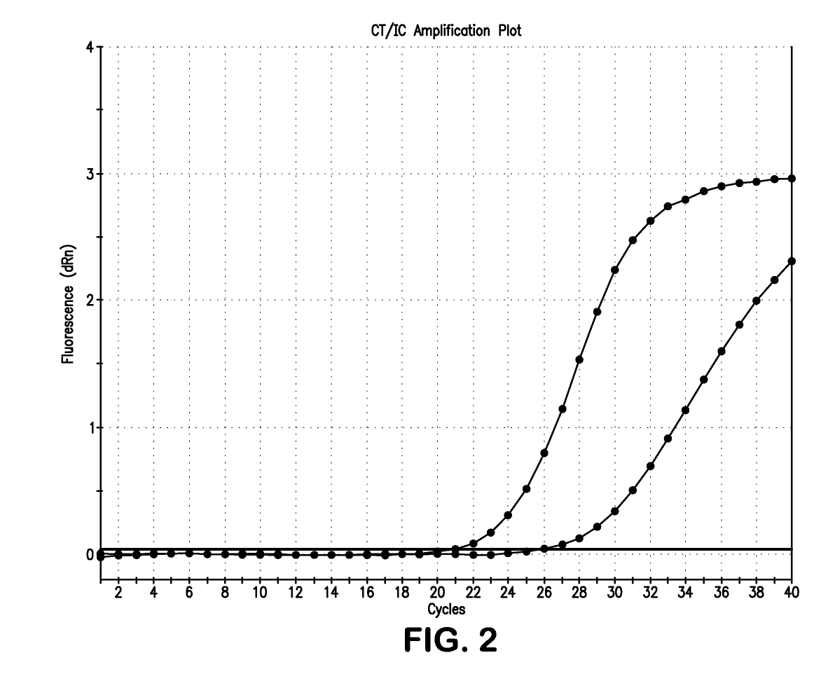

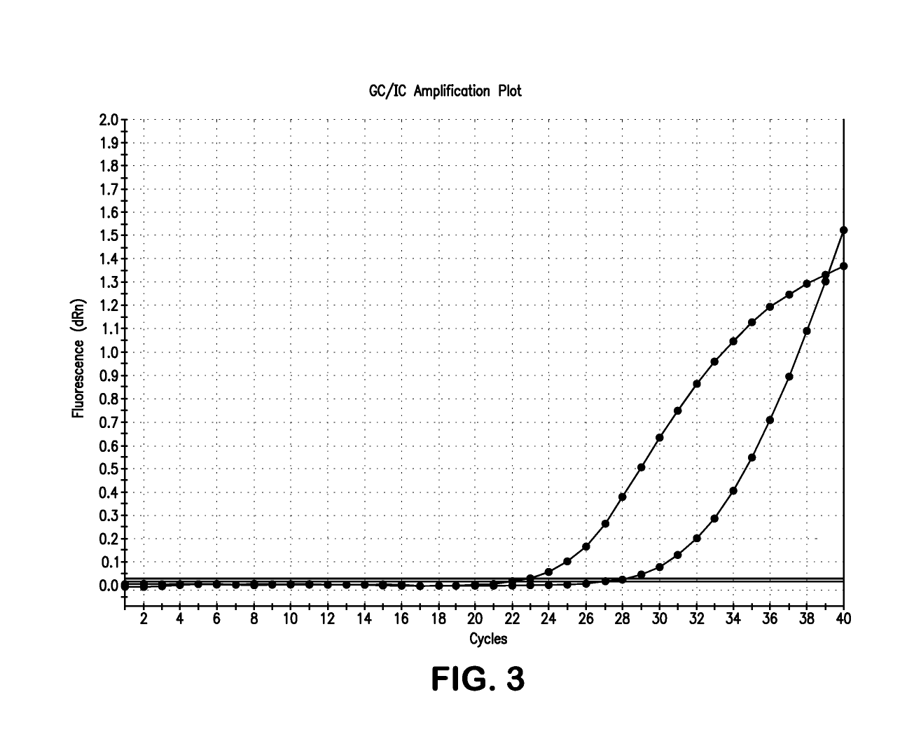

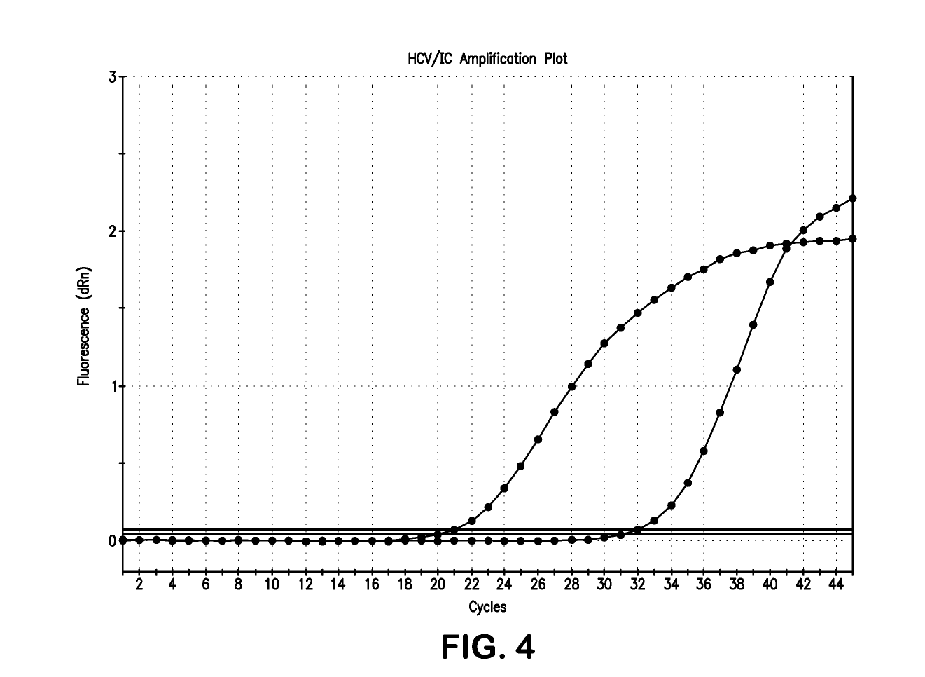

Independent RT-PCR assays were carried out with TAQMAN® probes (Applied Biosystems, Foster City, Calif.) for CT, GC, and HCV, respectively. Each assay included the target nucleic acid (DNA for CT and GC and RNA for HCV) and the MET IC of the present invention in a single well. FIGS. 2, 3, and 4 show the results of the amplification assays (cycle number versus delta Rn). Rn, the normalized reporter signal, is the fluorescence signal of the reporter dye divided by the fluorescence signal of the internal reference dye. Delta Rn (dRn) is determined by the formula Rⁿ⁺—Rⁿ⁻, where Rⁿ⁺ is the Rn value for a reaction involving all components and Rⁿ⁻ is the value for an unreacted sample. In each graph, the curve on the left represents amplification of the target and the curve on the right represents amplification of the IC.

We claim:

1. A non-competitive internal control for use in nucleic acid tests (NATs), comprising a nucleic acid obtained from an organism selected from Methanobacterium thermoautrophicum (MET) or Zea mays.

2. The non-competitive internal control of claim 1, wherein the nucleic acid is DNA.

3. The non-competitive internal control of claim 2, wherein the NATs are selected from the group consisting of: Influenza A, Influenza B, parainfluenza viruses Ito 4 (PIV-1 to PIV-4), respiratory syncytial virus type A (RSV A), RSV B, human metapneumovirus (hMPV), Hepatitis B virus (HBV), Chlamydia trachomatis (CT), and Neisseria gonorrhea (GC).

4. The non-competitive internal control of claim 1, wherein the nucleic acid is RNA.

5. The non-competitive internal control of claim 4, wherein the diagnostic NATs are selected from the group consisting of Hepatitis C virus (HCV), Human Immunodeficiency Virus 1 (HIV-1), and Severe Acute Respiratory Syndrome (SARS).

6. The non-competitive internal control of claim 1, wherein the nucleic acid comprises at least at least two primer binding sites and at least one probe binding site.

7. A method of preparing a non-competitive internal control for use in nucleic acid tests (NATs), comprising the steps of:
(a) extracting genomic DNA from Methanobacterium thermoautrophicum (MET);
(b) generating an amplicon from the genomic DNA of step (a) using forward and reverse primers having at least two restriction enzyme sites and a target specific sequence;
(c) generating a plasmid by ligating the amplicon of step (b) with a vector sequence having a promoter sequence and restriction enzyme sites that are identical to the restriction enzyme sites of the amplicon;
(d) digesting the plasmid with restriction enzymes corresponding to the restriction enzyme sites of steps (b) and (c) to generate MET internal control DNA.

8. The method of claim 7, further comprising the step of:
(e) preparing MET internal control RNA from the DNA of step (e).

9. The method of claim 7, wherein the at least two restriction enzyme sites of steps (b) and (d) correspond to the sequences of restriction enzymes XhoI and SpeI.

10. The method of claim 7, wherein the forward primer of step (b) has the sequence of SEQ ID. NO. 3 and the reverse primer of step (b) has the sequence of SEQ ID. NO. 4.

11. The method of claim 7, wherein the promoter sequence of step (c) is a T7 promoter sequence.

12. The method of claim 7, wherein the MET internal control DNA is used as an internal control in DNA nucleic acid diagnostic tests for the following disease states: Influenza A, Influenza B, parainfluenza viruses 1 to 4 (PIV-1 to PIV-4), respiratory syncytial virus type A (RSV A), RSV B, human metapneumovirus (hMPV), Hepatitis B virus (HBV), Chlamydia trachomatis (CT), and Neisseria gonorrhea (GC).

13. The method of claim 8, wherein the MET internal control RNA is used as an internal control in RNA nucleic acid diagnostic tests for the following disease states: Hepatitis C virus (HCV), Human Immunodeficiency Virus I (HIV-1), and Severe Acute Respiratory Syndrome (SARS).

14. A method of preparing a non-competitive internal control for use in nucleic acid tests (NATs) comprising the steps of:
(a) extracting genomic DNA from Zea Mays (Corn);
(b) generating an amplicon from the genomic DNA of step (a) using forward and reverse primers having at least two restriction enzyme sites and a target specific sequence;
(c) generating a plasmid by ligating the amplicon of step (c) with a vector sequence having a promoter sequence and restriction enzyme sites that are identical to the restriction enzyme sites of the amplicon;
(d) digesting the plasmid with restriction enzymes corresponding to the restriction enzyme sites of steps (b) and (c) to generate Corn internal control DNA.

15. The method of claim 14, further comprising the step of:
(e) preparing Corn internal control RNA from the DNA of step (e).

16. The method of claim 14, wherein the at least two restriction enzyme sites of steps (b) and (d) correspond to the sequences of restriction enzymes XhoI and SpeI.

17. The method of claim 14, wherein the promoter sequence of step (d) is a T7 promoter sequence.

18. The method of claim 14, wherein the Corn internal control DNA is used as an internal control in DNA nucleic acid diagnostic tests for the following disease states: Influenza A, Influenza B, parainfluenza viruses 1 to 4 (PIV-1 to PIV-4), respiratory syncytial virus type A (RSV A), respiratory syncytial virus type B (RSV B), human metapneumovirus (hMPV), Chlamydia trachomatis, Neisseria gonorrhea, and Hepatitis B virus (HBV).

19. The method of claim 15, wherein the Corn internal control RNA is used as an internal control in RNA nucleic acid diagnostic tests for the following disease states: Hepatitis C virus (HCV), Human Immunodeficiency Virus I (HIV-1), and Severe Acute Respiratory Syndrome (SARS).

20. The method of claim 14, wherein the forward primer of step (b) has the sequence of SEQ ID NO. 27 and the reverse primer of step (b) has the sequence of SEQ ID NO. 28.
